(19) United States
(12) Patent Application Publication
(10) Pub. No.: US 2010/0015657 A1
(43) Pub. Date: Jan. 21, 2010

(54) METHODS AND DEVICES FOR ASSESSING BIOLOGICAL FLUIDS

(75) Inventor: Jeffrey S. Dlott, Reston, VA (US)

Correspondence Address: FOLEY & LARDNER LLP, P.O. BOX 80278, SAN DIEGO, CA, 92138-0278, US

(73) Assignee: Quest Diagnostics Investments Incorporated

(21) Appl. No.: 12/506,056

(22) Filed: Jul. 20, 2009

Related U.S. Application Data
(60) Provisional application No. 61/082,480, filed on Jul. 21, 2008.

(51) Int. Cl. C12Q 1/02; C12M 1/34

(52) U.S. Cl. 435/29; 435/288.1; 435/287.9

(57) ABSTRACT

The present invention relates to devices, methods, and kits used to determine the source of an aliquot of a biological fluid, including the presence of additives in the biological sample.

### CROSS-REFERENCE TO RELATED APPLICATIONS

This application claims the benefit of U.S. Provisional Application No. 61/082,480, filed Jul. 21, 2008.

### FIELD OF INVENTION

The present invention relates to devices, methods, and kits for determining the presence of additives in a biological sample.

### BACKGROUND OF INVENTION

A variety of biological sample collection tubes are available in the medical field. Many of these biological sample collection, storage, and handling tubes contain additives in order to facilitate the preparation and/or storage of the collected biological fluid. For example, one type of common blood collection tube is designed for analysis of blood serum and is either completely empty or has a non-reactive gel at the bottom which allows for easy separation of the serum from cells by centrifugation. The separated serum is then used for a variety of tests, including chemistry, immunology, serology, and blood bank tests. Other types of blood collection tubes may contain one or more additives including, for example, a heparin salt to act as an anticoagulant, or a chelating agent (e.g., citrate or EDTA) to remove free ions from the blood sample. Heparin salts are typically added to blood samples in preparation for blood chemistry tests, citrate is typically added in preparation for blood coagulation tests, and EDTA is typically added for hematology and blood bank tests.

It is important to maintain and correctly track the biological samples containing these and other additives because certain additives can interfere with the results of certain analytical tests in which those additives are not intended. Errors in tracking the source of the samples arise during routine laboratory handling of large numbers or varying samples, particularly when a single sample obtained from a patient is designated for multiple assays or when multiple samples from the same patient are simultaneously processed for multiple assays. Typically, the collection container obtained from the clinician containing the original patient sample is labeled or otherwise coded indicating the type of sample and the additives, if any, that are present in the collection container. For blood tubes (e.g., Vacutainers®), this is typically done using color-coded stoppers. However, for routine laboratory analysis an aliquot of the biological fluid is typically removed from the original collection tube into a separate assay or preparatory container. Human error may result in the mislabeling of these secondary containers which receive the biological sample containing (or not containing) certain additives. A wrongly-labeled aliquot container is more prone to being processed incorrectly (i.e., tested with an assay affected by the presence of one or more routine additives), which can lead to inaccurate test results and/or delays in producing patient information. See, e.g., James, et al., AAPS Journal, 2007; 9(2) E123-127 (stating on p. E123 that “correct labeling and identification of a sample is critical; an ambiguously or incorrectly labeled sample will automatically result in an incorrect result.”); Lippi, et al., Int. Jnl. Lab. Hem., published online (stating on p. 3 that it is “exceedingly difficult to conclusively ascertain the definitive nature of the sample, given that samples collected with different additives would visually appear identical.”); and Bonini et al., Clinical Chemistry, (2002) 48, 691-698 (stating on p. 696 that evaluating submitted specimens is essential because “acceptance of improper specimens for analysis may lead to erroneous information that could affect patient care.”).

The present invention provides a biological sample collection tube and methods for determining the presence of certain additives in a biological sample. The inventive tubes may be used as secondary aliquot tubes, which may be interpreted by the user to indicate the additives present and/or the source of the biological sample.

### SUMMARY OF THE INVENTION

The invention relates to devices, methods, and kits used to evaluate the source of an aliquot of a blood sample (e.g., whole blood and blood derivatives such as serum and plasma), including determination of which, if any, routine additive are present. The invention provides inert substrates and reagents that undergo a visible color change indicative of the amount of certain ions present so that the source of the aliquot and/or the contained additives may be evaluated.

In one aspect, the invention provides a method for determining whether a patient requires retesting for a blood coagulation test comprising: (i) providing a blood sample having a prolonged clotting time result; (ii) assessing the free calcium concentration in said blood sample using a Ca-sensitive test strip; (iii) identifying the blood sample as having a valid result when the free calcium concentration is at least about 5 mg/dL; and (iv) identifying the blood sample as having an invalid result when the free calcium concentration is less than about 5 mg/dL.

In some embodiments, the method further comprises further identifying the blood sample as being collected in an EDTA-containing blood tube when the free calcium is less than about 1 mg/dL. In some embodiments, the method further comprises further identifying the blood sample as being collected in a blood tube containing no calcium chelator when the free calcium concentration is greater than about 7 mg/dL. In some embodiments, the method further comprises further identifying the blood sample as being collected in a citrate-containing blood tube when the free calcium concentration is between about 5 mg/dL and about 7 mg/dL. In some embodiments, the method further comprising further identifying said blood sample as a blood sample derived from an under-filled citrate tube when the free calcium concentration is between about 1 mg/dL and about 5 mg/dL.

In some embodiments, the blood coagulation test that is validated is a Factor VIII activity assay, a prothrombin clotting time assay, and a risocetin activity assay. For embodiments in which the coagulation test is a prothrombin clotting time assay, the blood sample provided optional has an INR≧about 4.0, 4.5, 5.0, 5.5, or 6.0.

Suitable calcium-sensitive test strips include, for example, Sofcheck® Water Quality Test Strips, and test strips containing arsenazo III or 3,3′-bis[N,N-di(carboxymethyl)aminomethyl]-o-cresolphthalein. In some embodiments, the blood sample is further contacted with a magnesium ion chelator such as, for example, 8-hydroxyquinoline and 8-hydroxyquinoline-5-sulfonate.

In other embodiments, the concentration of potassium in the blood sample is assessed using a potassium-sensitive test strip. Suitable potassium-sensitive test strips include potassium chelators such as valinomycin and 2,3-(naphtho)-15-crown-5. In some embodiments, the blood sample as being collected in an EDTA-containing blood tube when the free potassium concentration is greater than about 20 mM or in a blood tube containing no calcium chelator when the free potassium concentration is less than about 7 mg/dL.

In another aspect, the invention provides a sterile sample collection tube comprising a calcium-sensitive test strip, wherein said tube is suitable for receiving an aliquot of blood. Suitable calcium-sensitive test strips include, for example, Sofcheck® Water Quality Test Strips, and test strips containing arsenazo III or 3,3′-bis[N,N-di(carboxymethyl)aminomethyl]-o-cresolphthalein. In some embodiments, the blood sample is further contacted with a magnesium ion chelator such as, for example, 8-hydroxyquinoline and 8-hydroxyquinoline-5-sulfonate. Optionally, the tube further contains a magnesium ion chelator such as, for example, 8-hydroxyquinoline and 8-hydroxyquinoline-5-sulfonate, and/or a potassium-sensitive test strip. Potassium-sensitive test strips may contain potassium chelators such as valinomycin, 2,3-(naphtho)-15-crown-5. In some embodiments, the potassium test strip further contains a pH sensitive dye that undergoes a color change upon proton release (e.g., 4-[(2,6-dibromo-4-nitrophenyl)azo]-2-octadecyloxy-1-naphthol).

In another aspect, the invention provides a sterile container containing a single test strip having two ion-sensitive detection zone. Specifically, the test strip has: (a) a first region comprising a calcium ion chelator, and (b) a second region comprising a potassium ion chelator, wherein the first region and the second region are spatially distinct, said first region capable of undergoing a color change when exposed to calcium, said second region capable of undergoing a color change when exposed to potassium, and the test strip is affixed to an inner wall of said container and is visually observable from the exterior. Suitable calcium ion chelator include, for example, arsenazo III or 3,3′-bis[N,N-di(carboxymethyl) aminomethyl]-o-cresolphthalein. Suitable potassium ion chelators include, for example, valinomycin and 2,3-(naphtho)-15-crown-5 and the potassium-sensitive area may further contain a pH sensitive that undergoes a color change upon proton release (e.g., 4-[(2,6-dibromo-4-nitrophenyl)azo]-2-octadecyloxy-1-naphthol). The container optionally may further include a magnesium ion chelator (e.g., hydroxyquinoline or 8-hydroxyquinoline-5-sulfonate).

In another aspect, the invention provides a kit comprising any of the foregoing tubes or containers and a color guide calibrated to indicate the concentration of calcium based on a comparison with the calcium-sensitive test strip. In some embodiments, the color guide has a color gradient corresponding to at least three distinct calcium concentration ranges (e.g., less than about 1 mg/dL, between about 5 mg/dL and about 7 mg/dL, and greater than about 7 mg/dL.

In another aspect, the invention provides a method for evaluating an aliquot of a blood sample to determine the presence or identity of an additive that effects the level of at least one ion in the blood sample, comprising determining the concentration of one or more ions in said aliquot and identifying the additive based on the concentration of said one or more ions. In one embodiment at least one of the ions is calcium and the additive is identified as EDTA when said concentration of said calcium ion is less than 1 mg/dL. The calcium concentration may be determined using a calcium ion chelator such as, for example, arsenazo III or 3,3′bis[N,N-di(carboxymethyl)aminomethyl]-o-cresolphthalein. Optionally, the aliquot is further contacted with a magnesium ion chelator such as, for example, 8-hydroxyquinoline or 8-hydroxyquinoline-5-sulfonate. Optionally, the concentration of potassium is also assessed and the additive is identified as EDTA when said concentration of said calcium ion is less than 1 mg/dL and the concentration of said potassium is greater than about 20 mM.

In another embodiment, at least one of the ions is potassium and the aliquot is identified as being either additive-free or comprising heparin when said concentration of potassium is less than about 7 mM, or the additive is identified as citrate when said concentration of potassium is about 7-20 mM. The potassium concentration may be determined using a potassium ion chelator such as, for example, valinomycin or 2,3-(naphtho)-15-crown-5.

Although the invention has been characterized in terms of test strips (e.g., calcium-sensitive and potassium-sensitive test strips), it is understood that any suitable inert substrate may be substituted for one or more of the strips. Suitable substrates include, for example, tethered or untethered beads, membranes, resins, or polymers. In embodiments in which the inert substrates are untethered, it may be convenient to house those substrates (e.g., beads) in a separate compartment of the container or tube which is in fluid communication with the blood-containing compartment of that container or tube.

### DETAILED DESCRIPTION OF INVENTION

The present invention provides devices, methods, and kits used to evaluate the source of an aliquot of a blood sample (e.g., whole blood and blood derivatives such as serum and plasma), including determining which, if any, routine additive are present. In particular, the invention is useful for detecting levels of calcium and potassium ions in blood samples.

As used herein, “evaluating the source of a blood sample aliquot” indicates identifying the type of collection tube used to obtain, store, or transport a blood sample prior to aliquoting by identifying the presence and/or type of additives present in the blood sample alequot. For example, blood samples are routinely obtained from subjects using a variety of types of collection tubes which optionally contain one or more additives for storage and/or processing. Blood collection tubes include, for example, untreated (i.e., “serum”) tubes, heparinized tubes, citrate tubes, and EDTA tubes. It is not readily apparent to the clinician what additives, if any, have been introduced into the sample once the sample has been aliquoted from the original collection tube into secondary (e.g., assay, aliquot, or storage) tubes. The presence and nature of an additive in the original collection tube may be determined using the methods and devices described herein, thereby “identifying the source of a biological sample aliquot.”

As used herein, the term “inert substrate” indicates a substrate which is not reactive with the sample being tested, and does not necessarily indicate a lack of reactivity with reagents incorporated on or into the substrate. Thus, an inert substrate may be a substrate capable of covalently bonding to one or more chemical reagents.

As used herein, the term “calcium ion chelator” indicates a compound that binds one or more calcium ions with dissociation constant of at least about 10⁻⁵ M, 10⁻⁶ M, 10⁻⁷ M, 10⁻⁸ M, 10⁻⁹ M, 10⁻¹⁰ M, 10⁻¹² M, or less.

As used herein, the term “potassium ion chelator” indicates a compound that binds one or more potassium ions with dissociation constant of less than or equal to about 10⁻⁵ M, such as about 10⁻⁶ M, 10⁻⁷ M, 10⁻⁸ M, 10⁻⁹ M, 10⁻¹⁰ M, 10⁻¹² M, or less.

As used herein, the term “magnesium ion chelator” indicates a compound that preferentially binds magnesium ions over calcium ions by about 5-fold or more, such as about 10-fold, 50-fold, 100-fold, 1000-fold, or more.

As used herein, the term “ion-sensitive test strip” (e.g., “calcium-sensitive test strip” and “potassium-sensitive test strip”) refers to a inert substrate that contains a chelator or other ion binding compound which undergoes a visible color change when the target ion (e.g., calcium or potassium) is bound.

As used herein, the term “visually observable” as a description of the physical orientation of a test strip within a biological sample container indicates that the test strip is oriented in such as way as to permit an observer to visually determine a color change on the test strip from the exterior of the container.

As used herein, the term “bead” indicates any discrete form of an inert substrate that may be coated with or otherwise contain reagents capable of undergoing a color change in the presence of an analyte of interest. Beads may have any convenient shape or size; preferably beads are adapted to be visually observable once inserted into a container (e.g., a blood tube or aliquot tube).

As used herein, the term “citrate tube” is used to indicate a blood collection container into which a portion of citrate buffer has been added. Preferably, the citrate is contained within the unused tube as a dry reagent coated on the inner container walls. Citrate tubes are widely known and used in the art (e.g., Becton Dickenson, Vacutainer Plus®; catalog no. 363080).

As used herein, the term “blood sample derived from an under-filled citrate tube” indicates a sample of blood (including an aliquot) which contains an inappropriately high concentration of citrate. Most commonly, blood samples with inappropriately high citrate concentrations are generated when a less than a full portion of blood is collected in a citrate-containing collection tube. The artisan understands that the additive-containing collection tubes contain a pre-measured amount of the additive (e.g., citrate) intended for dilution in a full portion of blood intended for collection in that tube. When less than a full portion of blood is collected the additive is present at a higher concentration than expected or intended, which may lead to inaccurate assay results. In some embodiments, the citrate tube is under-filled by at least 10%, such as by at least 20%, 30%, 40%, 50%, 60%, 70%, 80%, 90%, or more. That is, the amount of blood collected in the citrate tube is less than or equal to 90% of the appropriate amount, such as less than or equal to about 80%, 70%, 60%, 50%, 40%, 30%, 20%, 10%, or less.

As used herein, the term “heparin tube” is used to indicate a blood collection container into which a portion of heparin, or a heparin salt which inhibits blood coagulation, has been added. Preferably, the heparin is contained within the unused tube as a dry reagent coated on the inner container walls. Heparin tubes are widely known and used in the art (e.g., Becton Dickenson, Vacutainer Plus®; catalog no. 367871).

As used herein, the term “EDTA tube” is used to indicate a blood collection container into which a portion of EDTA, or an EDTA salt, has been added. Preferably, the EDTA is contained within the unused tube as a dry reagent coated on the inner container walls or contained within a polymer gel. EDTA tubes are widely known and used in the art (e.g., Becton Dickenson, Vacutainer Plus®; catalog no. 362788).

As used herein, the term “untreated tube” is used to indicate a blood collection container which does not contain heparin, citrate, or EDTA.

As used herein, the use of terms “about” or “approximately” in reference to a numerical value refers to an inclusive range of the indicated numerical value±10%.

### Inert Substrates

The invention provides a variety of inert substrates that contain reagents and/or reagent systems which undergo a visual color change upon exposure to certain ions. The inert substrates may consist of any inert material(s) capable of retaining the reagents and adapted for the desired application. For example, inert substrates include test strips, beads or other untethered objects that may be placed within a container, or may comprise the inner wall of a liquid storage container or reaction vessel. The inert substrates may be constructed from paper (e.g., filter paper), cotton, hydrophobic or hydrophilic membranes, or thermoplastic resins and polymers.

Inert substrates may be prepared from a variety of suitable starting materials selected based on the specific application (i.e., type of biological fluid and style of container) for which the test strips will be used, as well as the specific nature of the reagents that will be contained within the substrates. Other considerations in the choice of material for the inert substrates include physical properties such as thickness, absorbency, particle retention, wicking speed, and the like. In embodiments where the inert substrate is in the form of a test strip, suitable test strip materials include, for example, pre-made cotton fiber dipsticks (Model CF3; Whatman) and a variety of membranes and filter papers such as nitrocellulose (Optitran nitrocellulose membrane; Whatman), PVDF, GFTE, and nylon membranes, or any number of filter papers.

Additionally, substrates may be laminated and contain more than one material, depending upon the technical needs of the detection reagents. A single substrate may also be formed from multiple materials with different polarities, in order to provide a polar phase and a non-polar phase on the same substrate.

Generally, one or more chemical reagents may be incorporated on or into a substrate by applying liquid solutions of the reagents to the substrate and allowing the reagent solutions to dry. In certain embodiments, it may be necessary to chemically attach the reagents to the substrates. Attachment may be facilitated by covalent bonding of the reagents to a substrate. The particular attachments are dictated by the specific chemistry of the reagent and the reactivity of the selected substrate. For example, a non-polar substrate may optionally be treated with a surfactant or the like in order to immobilize the polar reagents.

Reagent containing substrates may contain additional reagents which enhance performance. For example, reagent containing substrates may also contain one or more mordants, binders, buffers, and ionic chelators that enhance to sensitivity and/or specificity of the detection reagents. One particularly useful binder is polyvinyl pyrrolidone.

### Calcium-Reactive Substrates

Calcium-reactive test strips may be formed by impregnating a substrate with a calcium ion chelator that undergoes a detectible change upon exposure to calcium ions. Preferably the change is visibly detectible to the human eye. A number of such calcium ion chelators are known in the art and may be used, including arsenazo III or 3,3′-bis[N,N-di(carboxymethyl)aminomethyl]-o-cresolphthalein (hereinafter “cresolphthalein”).

Many potentially useful calcium chelators also chelate other ions, particularly other divalent cations. Thus, in some embodiments, one or more visually undetectable chelators with a higher specificity for potentially interfering cations may additionally be incorporated on or into a calcium chelator containing substrate. These additional chelators improve sensitivity and specificity in the performance of the detectable calcium chelator by binding non-calcium cations, and thereby diminishing or removing possible sources of interference. For example, cresolphthalein undergoes a visually detectible change upon chelating either calcium or magnesium. Thus, cresolphthalein may be used in combination with a magnesium chelator such as 8-hydroxyquinoline (U.S. Pat. No. 4,594,225). Inclusion of a magnesium chelator reduces the amount of magnesium available for cresolphthalein to bind, and thus reduces false signal from cresolphthalein bound to magnesium. Other useful chelators that preferentially bind magnesium ions over calcium ions include, for example, 8-hydroxyquinoline-5-sulfonate.

Other non-chelating reagents may be incorporated on or into a substrate in order to affect the substrate's local chemical environment. Specifically, a non-chelating reagent that affects the pH of the substrate may be incorporated. The effect on the substrate pH may be to increase pH, decrease pH, or to act as a pH buffer (that is, to stabilize the pH at or near some preferred value). The necessity and nature of any such reagent is highly specific to the chemistries of the chelating reagents used. For example, the cresolphthalein and 8-hydroxyquinoline containing test strip discussed above may additionally contain Na₃PO₄.H₂O in order to maintain the test strip in a highly basic state. Inclusion of this additional reagent is beneficial to this particular combination of indicators because the performance of 8-hydroxyquinoline (U.S. Pat. No. 4,594,225) increases at high pH. However, other chelating reagent combinations may perform better under different pH conditions.

### Potassium-Reactive Substrates

Potassium-reactive substrates may be manufactured in a similar manner as described for calcium-reactive substrates and contain potassium ion chelators that undergo a color change (preferably in the visible range) upon exposure to potassium. In some embodiments, a potassium ion chelator is used in association with a pH sensitive dye. Generally, the potassium ion chelator and the pH sensitive dye are closely associated on the substrate, preferably in the same spatial location. Commonly, co-localization of the potassium ion chelator will be achieved by mixing the liquid reagents for a single application to the substrate during preparation. Alternatively, the two compounds may be added sequentially during preparation of the substrate, with an optional drying step in between each application.

In one example, the potassium chelator is valinomycin and the pH indicator dye is 4-[(2,6-dibromo-4-nitrophenyl)azo]-2-octadecyloxy-1-naphthol. Both reagents are contained within an ion-permeable non-polar phase of the substrate. Valinomycin chelates a potassium ion, causing a net positive charge in the non-polar phase. The pH indicator dye releases a proton from the non-polar phase into the polar phase, wherein this proton loss causes a color change. Ng, et al. Clin. Chem. 38(7), pp. 1371-1372 (1992). Valinomycin test strips are commercially available as Reflotron K⁺™ (Roche Diagnostics). Other potassium-selective ionophores, such as 2,3-(naphtho)-15-crown-5 (Ames Seralyzer™ potassium strips) can be used to mediate proton release from pH indicator dyes. Gibb, J. Clin. Pathol. 1987; 40, p. 298.

### Determination of Test Strip Results

In order to determine the source of the aliquot of the biological sample, it is necessary for the user to determine the concentration of calcium and or potassium in the aliquot of the blood sample. This is accomplished by visual observation of a test strip in the presence of the sample. The test strip undergoes a color change proportionate to quantity of calcium and/or potassium present in the sample. For example, a test strip containing cresolphthalein, which is sensitive to calcium ions, will reflect light at a wavelength of about 575 nm and appear yellow to the eye. The greater the concentration of calcium ions in the biological sample, the more intensely yellow the test strip will appear. Similarly, a test strip containing the combination of valinomycin and 4-[(2,6-dibromo-4-nitrophenyl)azo]-2-octadecyloxy-1-naphthol will turn red in the presence of potassium.

In some embodiments, interpretation of the degree of color change is aided by the use of a color guide which correlates the observed color of the test strip with an approximate concentration of the analyte of interest. The color guide shows a continuous or discreet color gradient, wherein various colors on the gradient are identified to correspond approximately to the color exhibited by a test strip in the presence of a known analyte concentration. Analyte concentrations associated to colors on the gradient may be provided as unique values or ranges. Color guides may be prepared for any colored reagent incorporated on or in the test strip. Color guides are usually prepared empirically by testing a batch of test strips at several known concentrations of analyte and constructing a guide based on the resulting information.

Color guides may be present as a component separate from a test strip in a sample container (e.g., provided as a package insert). Alternatively, the color guide may be affixed to the outer wall of the sample container with which it is associated. In the latter configuration the color guide is preferably affixed adjacent to the test strip to which it refers to facilitate visual comparison with the test strip.

Determination of a Primary Blood Container Type from Blood Aliquot

As described above, primary blood collection containers may contain additives such as citrate, EDTA, or heparin. These common additives to primary blood collection tubes act to varying degrees as calcium and/or potassium chelators. Thus, the presence or absence of these additives in a blood sample may be determined based on the free calcium and/or free potassium concentration remaining in the blood aliquot.

For example, EDTA is a strong calcium chelator. Thus, blood aliquots having a calcium concentration of less than about 2 mg/dL may be identified as being derived from an EDTA tube, whereas calcium concentration of greater than about 2 mg/dL may be identified as being derived from some other, non-EDTA, tube.

The additive EDTA is most commonly present as a potassium salt. Thus, in some embodiments, blood aliquots identified as being derived from an EDTA tube may be further identified as having a potassium concentration greater than about 20 mM, such as greater than about 25 mM, such as greater than about 30 mM.

Citrate also chelates calcium, but to a much lesser extent than EDTA. Thus, blood aliquots derived from a citrate tube have a calcium concentration within the range of about 2 mg/dL to about 7 mg/dL, such as in the range of about 5 mg/dL to about 7 mg/dL. Heparin also chelates calcium, but to a lesser extent than citrate. Thus, blood aliquots derived from a heparin tube have a calcium concentration within the range of about 6 mg/dL to about 11 mg/dL. Table 1 shows typical calcium and potassium concentration ranges from various primary collection tubes.

TABLE 1
Typical Calcium and Potassium Concentration Ranges

| | EDTA | Citrate | Heparin | Serum |
|---|---|---|---|---|
| | Tube | Tube | Tube | (untreated tube) |
| Calcium (mg/dL) | <<2 | 5-7 | 6-11 | 8-10 |
| Potassium (mM) | 25-41 | 7.5-15.5 | 3.5-5.5 | 3.5-5.5 |

Based on the foregoing calcium and potassium concentrations, color guides may be constructed as indicators of calcium and/or potassium levels in blood aliquots. A color guide for calcium may be constructed having a continuous or discontinuous color gradient. One or more cutoff values may be conveniently indicated. One suitable cutoff value is indicated on the color guide, for example, at about 2 mg/dL. The cutoff value may be modified and or additional cutoff values may be added depending upon the specific application for which the sample container is used. For blood testing, it is expected that aliquots derived from EDTA tubes will contain calcium concentrations nearly an order of magnitude below the 2 mg/dL cutoff value, whereas aliquots derived from non-EDTA tubes will have calcium concentrations more than two-fold higher than the 2 ng/dL cutoff value. This characteristic should facilitate easy identification of the presence of EDTA in the source blood tube by the user.

Also based on the foregoing, a color guide may be constructed having a continuous or discontinuous color gradient indicative of potassium concentration. Here again, one or more cutoff values may be conveniently indicated on the color guide. One suitable cutoff value is indicated on the color guide, for example, at about 7 mM. Optionally, a second cutoff value is indicated at about 20 mM, 25 mM, or 30 mM. These values are expected to provide a color guide with sufficient resolution to provide the user with further distinction of citrate source tubes and heparin or untreated source tubes on the low end, and EDTA source tubes on the high end.

Identification of Blood Aliquots Obtained from Under-Filled Citrate Tubes

Blood clotting time is a widely used measurement and is frequently performed on blood samples collected in citrate tubes (i.e., blood samples to which citrate has been added). One problem that arises in accurately determining blood clotting time arises from the use of blood from under-filled citrate tubes. Blood from under-filled citrate tubes has an excess amount of citrate in the blood sample which results in chelation of more calcium than properly filled citrate tubes. The disproportionate reduction in free calcium leads to prolonged clotting times in the blood sample, yielding inaccurate results.

In situations where a blood sample is known to have come from citrate tube, but confirmation that the citrate tube was appropriately filled at collection is desired, the fill status of the citrate tube may be derived by determination of the level of calcium in the blood sample by using a test strip as described herein. An inappropriately low calcium level is indicative of an under-filled citrate collection tube. For example, as shown in Table 1, it is expected that the calcium concentration in blood samples collected in citrate tubes is about 5-7 mg/dL. Calcium concentrations below this level (e.g., less than about 5, 4, 3, 2, or 1 mg/dL) are identified as being derived from under-filled citrate tubes.

Once the sample has been properly identified as being derived from an under filled tube, the measured clotting time either be disregarded or empirically adjusted based on the estimated calcium level.

### Container Construction

The test strip(s) must be in fluid communication contact with the lumen of the container in order that the ions of interest can be detected in the biological fluid. Typically, the test strip(s) are affixed to the inner wall of the container and/or the reagents are impregnated into or coated on the inner wall of the container. In one embodiment, the container is made of a material that is sufficiently transparent or translucent in order that the color change reaction(s) on the test strips can be observed by the user from the exterior of the container.

In some embodiments, it may be necessary that the container be relatively impervious to light (especially ultraviolet light) in order to prevent the biological sample and/or assay reagents from photodegradation. In these cases, the container may be constructed from an opaque or semi-opaque material, but contain a transparent or translucent “window” through which the test strip can be viewed.

The test strips may be affixed by any appropriate means to the inner wall of the container. In one embodiment, the inner wall contains a channel or groove into which the test strip fits such that the entire reagent strip is in liquid contact with the contents of the container. In another embodiment, the test strip is contained within an enclosed channel such that only a portion (e.g., a terminal portion) of the test strip is in direct fluid communication with the biological sample. Alternatively, a portion of the test strip that does not contain an ion chelator contains a layer of porous material, while the remainder of the test strip is coated with a clear, impermeable coating. In either configuration, the analyte-containing fluid is drawn through the test strip by capillary action with the origin being the portion of the test strip actually in fluid communication with the biological sample.

Optionally, the test strip is separated from the biological sample by a porous material. For example, the porous material may be configured to permit the passage of the analytes of interest (e.g., calcium and potassium ions) while excluding larger debris and/or interfering substances such as cells and other particulate matter. Such porous material can be glass fiber filter paper such as Multigrade GMF 150 glass fiber filters, or Nuclepore® track-etched polycarbonate membranes (both commercially available from Whatman). Nuclepore® membranes are available with a range of pore sizes from 0.015 microns to 12 microns, while Multigrade GMF 150 glass fiber filters can retain particles 1.2 microns and bigger or 2.5 microns and bigger (depending on the model). PVDF and other membranes having an average pore size of 0.45 μm or less are also useful.

The following examples serve to illustrate the present invention. These examples are in no way intended to limit the scope of the invention.

### EXAMPLES

### Example 1

### Manufacture of a Calcium-Sensitive Test Strip

A pre-formed cotton dipstick (Whatman, model CF3) is used as the basis for a test strip. An aqueous solution of 3,3′-bis[N,N-di(carboxymethyl)aminomethyl]-o-cresolphthalein is prepared at a concentration of about 10-1000 μM. The dipstick is dipped into the cresolphthalein solution for a time sufficient to saturate the test strip with solution. The dipstick is air-dried in an inverted position to allow excess solution to drain. The dried dipstick is shaped to form a test strip that conforms to the inner wall of the container into which the test strip will be placed.

### Example 2

### Manufacture of a Potassium-Sensitive Test Strip

A PVDF membrane is used as the basis for a test strip. An aqueous solution of valinomycin (about 10-1000 μM) and 4-[(2,6-dibromo-4-nitrophenyl)azo]-2-octadecyloxy-1-naphthol (about 10-1000 μM) is prepared. The PVDF membrane is treated with a surfactant to facilitate attachment of the reagents and then dipped into the cresolphthalein solution for a time sufficient to saturate the test strip with solution. The PVDF membrane is air-dried in a horizontal position in order to ensure an even coating of reagents on the membrane. The dried membrane is shaped to form a test strip that conforms to the inner wall of the container into which the test strip will be placed.

### Example 3

### Instrumental Determination of Calcium and Potassium Concentration Ranges in Various Blood Sample Types

Various serum, citrate plasma, EDTA plasma, and heparin plasma samples were analyzed for calcium concentration on an Olympus AU5400 Chemistry Analyzer according to the Arzenazo III Dye method. In the following Examples, blood samples were collected in the Becton Dickenson Vacutainers® and are identified as follows:

| Designation | Stopper color | Additive |
|---|---|---|
| EDTA Plasma | purple/lavender | potassium EDTA |
| Serum | red | no chelator |
| Citrate Plasma | light blue | sodium citrate |
| Heparin Plasma | green | sodium or lithium heparin |

In brief, this assay system reacted the arzenazo III dye with calcium in an acidic solution to produce a blue-purple complex. The colored solution was measured spectrophotometrically at a wavelength of 660 nm.

Similarly, the various sample types were analyzed for potassium as well. The resulting data is presented in Tables 2 and 3 below.

TABLE 2
Measured Calcium Ranges

| Sample Type | Calcium Range (mg/dL) |
|---|---|
| Serum | 8.6-10.2 |
| Heparin Plasma | 6.25-10.51 |
| Citrate Plasma | 5.6-6.9 |
| EDTA Plasma | 0.21-0.68 |

TABLE 3
Measured Potassium Ranges

| Sample Type | Potassium Range (mM) |
|---|---|
| Serum | 3.5-5.3 |
| Heparin Plasma | 4.33 (SD ± 0.11) |
| Citrate Plasma | 11.9 (SD ± 2.3) |
| EDTA Plasma | 33 (SD ± 4.0) |

### Example 4

### Identification of EDTA Plasma, Citrate Plasma, and Serum Samples

The calcium concentration of known sample types (serum, potassium EDTA, and sodium citrate) was tested using commercially available Sofcheck Water Quality Test Strips, manufactured by Hach Co, Loveland, Colo., and quantified using an Olympus AU5400 Chemistry Analyzer. The results are presented in Table 4.

The Sofcheck strips allow for the semi-quantitative determination of calcium concentration with an indication of high calcium concentration (red on the test strip indicating about greater than 250 ppm), medium calcium concentration (brown on the test strip indicating about 120 ppm), and low calcium concentration (green on the test strip indicating less than about 1.5 ppm).

TABLE 4
Identification of EDTA Plasma, Citrate Plasma, and Serum Samples

| Sample Type | Measured Calcium (mg/dL) | Test Strip Result |
|---|---|---|
| Serum #1 | 7.4 | Red |
| Serum #2 | 10.1 | Red |
| Serum #3 | 9.9 | Red |
| Serum #4 | 8.6 | Red |
| Serum #5 | 8.0 | Red |
| Serum #6 | 9.0 | Red |
| Serum #7 | 10.7 | Red |
| Serum #8 | 8.8 | Red |
| Serum #9 | 11.1 | Red |
| Serum #10 | 10.3 | Red |
| EDTA (54812150) | 0.04 | Green |
| EDTA (54813212) | −0.87 | Green |
| EDTA (54812248) | −0.87 | Green |
| Citrate (54788766) | 6.24 | Brown |
| Citrate (54809733) | 5.91 | Brown |
| Citrate (54802117) | 6.35 | Brown |

As seen above, a clear distinction in calcium concentration between blood samples collected in citrate plasma, EDTA plasma, and serum tubes was obtained with the test strip method. The test strip results were confirmed using by quantitative calcium analysis.

### Example 5

### Confirmation of EDTA Plasma for Factor VIII Activity Samples

Factor VIII-dependent clotting activity is a calcium-dependent process. Accordingly, a Factor VIII activity assay is typically performed on blood samples collected in citrate tubes because, although citrate chelates some free calcium, sufficient free calcium is available to accurately determine Factor VIII activity. Normal values are typically about 50%-200%. However, Factor VIII activity assays performed on blood samples collected in EDTA tubes are expected to yield erroneous results, with values of <1%. The erroneous results are believed to arise for the lack of available free calcium as a result of the high chelating capacity of EDTA, Likewise, the ristocetin cofactor assay is used as a calcium-dependent indicator of von Willebrand factor (vWF) activity. Therefore, when an abnormal result is obtained in any calcium-dependent assay, verification of sample type should be is performed before the value is released from the laboratory. The verification is routinely conducted via a calcium assay. Similarly, samples with other unusual clotting based results (i.e., factors) are tested as well.

Ten samples which demonstrated unusual clotting based results (including critical Factor VIII values) were tested with commercially available Sofcheck Water Quality Test Strips and an Olympus AU5400 Chemistry Analyzer for comparison. Results of these tests are presented in Table 5.

TABLE 5
Identification of Plasma Type for Samples Exhibiting Unusual Clotting Results

| | | Measured Calcium | Test Strip | Conclusion |
|---|---|---|---|---|
| Clotting Test and Result | | (mg/dL) | Result | (sample type) |
| Factor VIII (#2847) | less than 1% | 0.23 | Green | EDTA sample |
| Factor VIII (#8313) | less than 1% | 0.4 | Green | EDTA sample |
| Factor VIII (#8676) | ~9% | | Brown | Citrate sample |
| Factor VIII (#7429) | ~24% | 6.4 | Brown | Citrate sample |
| Thrombin Time | 34 seconds | 6.0 | Brown | Citrate sample |
| (#5948, tube #1) | (normal = less | | | |
| | than 23 seconds) | | | |
| Thrombin Time | 17.8 seconds | 6.2 | Brown | Citrate sample |
| (#5948, tube #2) | (normal = less | | | |
| | than 23 seconds) | | | |
| Factor VIII (#2261) | 20% | 5.8 | Brown | Citrate sample |
| Factor VIII (#8614) | 28% | 6.1 | Brown | Citrate sample |
| Factor VIII (#8888) | 20% | 6.4 | Brown | Citrate sample |
| Ristocetin (#9999) | <20% | 6.9 | Brown | Citrate sample |

Thus, the data indicate that the test strips are useful to differentiate sodium citrate plasma from EDTA plasma for plasma samples demonstrating unusual clotting behavior. For example, sample nos. 2847 and 8313 should not be reported and the patients should be retested because the abnormally low Factor VIII activity readings may be a result of the blood sample being collected in an inappropriate tube for the assay. Specifically, these samples may have blood collected in EDTA tubes. However, the possibility that the patient is hypocalcemic should also be investigated. In contrast, sample nos. 8676, 7429, 2261, 8614, 8888, and 9999 are most likely valid results identifying patients having impaired clotting ability. The patient from which sample no. 5948 was drawn should likely be retested because the thrombin time results are equivocal but the blood sampling protocol appears to be appropriate with respect to the chelating agent.

### Example 6

### Identification of Underfilled Citrate Tubes Following Prothrombin Time Test

The prothrombin time (PT) test measures the time for clot formation in a blood sample. During clot formation, prothrombin is converted to thrombin, which is one of the last steps in the clotting cascade. The PT test evaluates the integrated function of the coagulation factors that make up the extrinsic and common pathways of the coagulation cascade which includes Factors I, II, V, VII, and X. Deficiency in the amount or function of any of these factors will be observed as a prolonged clotting time in the PT.

The clotting time measured in the PT is known to have both inter-laboratory and intra-laboratory variability based on the specific reagents used. Additional variability is observed in blood samples obtained from patients receiving anti-coagulation therapy such as blood-thinning agents (e.g., Coumadin®). In order to standardize PT results, most laboratories have adopted the World Health Organization recommendations for use of the Internationalized Normalized Ratio (INR) which adjusts for changes in the PT reagents (i.e., normalize for inter- and intra-laboratory variability) and to evaluate PT results for patients on blood-thinning medications. Typically, patients on blood-thinning medications should have an INR of about 2.0-3.0, but ranges as high as about 2.5-3.5 may be appropriate for patients at particularly high risk of clot formation. Prolonged clotting time in individuals not on blood-thinning medications may be indicative of a variety of conditions including liver disease or vitamin K deficiency. However, as for other clotting time assays, the PT assay is calcium-dependent. The PT assay is typically performed on blood samples collected in citrate-containing blood collection tubes. The PT assay is therefore sensitive to under-filled collection tubes which results in an inappropriately high concentration of citrate in the blood sample, thereby chelating more free calcium than intended, which may give rise to a prolonged clotting time in a normal sample.

To investigate the prevalence and effects of under-filled citrate tubes, blood samples yielding abnormal INR (INR ≧5.0) results were identified over five consecutive working days and further analyzed for possible causes of a spurious result. As shown in Table 6, a significant proportion of blood samples yielding abnormal INR values during the study period were obtained from under-filled citrate tubes.

TABLE 6
Identification of Under-filled Citrate Tubes in Blood Samples
Having Abnormal INR Values Over a Five-day Study Period

| | # of Samples | | | |
|---|---|---|---|---|
| Study | Having | # of Under- | # of Expired | # of Tubes Showing |
| Day | INR ≧5 | filled Tubes | Tubes | Hemolysis |
| Day 1 | 38 | 2 | 2 | 0 |
| Day 2 | 33 | 6 | 3 | 0 |
| Day 3 | 40 | 4 | 2 | 1 |
| Day 4 | 37 | 6 | 3 | 1 |
| Day 5 | 26 | 1 | 3 | 0 |
| Total | 174 | 19 (11%) | 13 (7%) | 2 (1%) |

All data in Table 6, other than the INR values, were obtained by visual inspection of the original blood collection tubes obtained from the clinical laboratory. The patients from which the samples were collected in under-filled or expired tubes, or those showing hemolysis, may be indicated for re-testing because of the potential unreliability of the PT assay results. These data further indicate that, of the identifiable errors that possibly result in an incorrect determination of clotting time in the PT assay, collection tube under-filling by the phlebotomist is the most common.

What is claimed is:

1. A method for determining whether a patient requires retesting for a blood coagulation test comprising:
(i) providing a blood sample having a prolonged clotting time result in a blood coagulation test;
(ii) assessing the free calcium concentration in said blood sample using a calcium-sensitive test strip;
(iii) identifying the patient has not requiring retesting when the free calcium concentration in the sample is at least about 5 mg/dL; and
(iv) identifying the patient as requiring retesting when the free calcium concentration in the sample is less than about 5 mg/dL.

2. The method of claim 1, wherein the blood coagulation test is selected from the group consisting of a Factor VIII activity assay, a prothrombin clotting time assay, and a risocetin activity assay.

3. The method of claim 2, wherein the blood coagulation test is a prothrombin clotting time assay.

4. The method of claim 3, wherein the blood sample is identified as having an INR ≧4.0.

5. The method of claim 1, further comprising identifying said blood sample as being collected in an EDTA-containing blood tube when the free calcium concentration is less than about 1 mg/dL.

6. The method of claim 1, further comprising identifying said blood sample as being collected in a blood tube containing no calcium chelator when the free calcium concentration is greater than about 7 mg/dL.

7. The method of claim 1, further comprising identifying said blood sample as being collected in a citrate-containing blood tube when the free calcium concentration is between about 5 mg/dL and about 7 mg/dL.

8. The method of claim 1, further comprising identifying said blood sample as a blood sample derived from an under-filled citrate tube when the free calcium concentration is between about 1 mg/dL and about 5 mg/dL.

9. The method of claim 1, wherein the calcium-sensitive test strip is a Sofcheck® Water Quality Test Strip.

10. The method of claim 1, wherein the calcium-sensitive test strip comprises arsenazo III or 3,3′-bis[N,N-di(carboxymethyl)aminomethyl]-o-cresolphthalein.

11. The method of claim 1, wherein the blood sample is further contacted with a magnesium ion chelator.

12. The method of claim 11, wherein the magnesium ion chelator is 8-hydroxyquinoline or 8-hydroxyquinoline-5-sulfonate.

13. The method of claim 1, further comprising determining the concentration of potassium in the blood sample using a potassium-sensitive test strip.

14. The method of claim 13, wherein said potassium-sensitive test strip comprises valinomycin and 2,3-(naphtho)-15-crown-5.

15. The method of claim 13, further comprising further identifying said blood sample as being collected in an EDTA-containing blood tube when the free potassium concentration is greater than about 20 mM.

16. The method of claim 13, further comprising further identifying said blood sample as being collected a blood tube containing no calcium chelator when the free potassium concentration is less than about 7 mg/dL.

17. A sterile sample collection tube comprising a calcium-sensitive test strip, wherein said tube is suitable for receiving an aliquot of blood.

18. The tube of claim 17, wherein the calcium-sensitive test strip is a Sofcheck® Water Quality Test Strip.

19. The tube of claim 17, wherein the calcium-sensitive test strip comprises arsenazo III or 3,3′-bis[N,N-di(carboxymethyl)aminomethyl]-o-cresolphthalein.

20. The tube of claim 17, further comprising a magnesium ion chelator.

21. The tube of claim 20, wherein the magnesium ion chelator is selected from the group consisting of 8-hydroxyquinoline and 8-hydroxyquinoline-5-sulfonate.

22. The tube of claim 17 further comprising a potassium-sensitive test strip.

23. The tube of claim 22, wherein said potassium-sensitive test strip comprises valinomycin and 2,3-(naphtho)-15-crown-5.

24. The tube of claim 23, wherein said potassium-sensitive test strip comprises a pH sensitive dye that undergoes a color change upon proton release.

25. The tube of claim 24, wherein said pH sensitive dye is 4-[(2,6-dibromo-4-nitrophenyl)azo]-2-octadecyloxy-1-naphthol.

26. A sterile container comprising a test strip, wherein said test strip comprises:
(a) a first region comprising a calcium ion chelator, and
(b) a second region comprising a potassium ion chelator,
wherein said first region and said second region are spatially distinct, said first region capable of undergoing a color change when exposed to calcium, said second region capable of undergoing a color change when exposed to potassium, and said test strip is affixed to an inner wall of said container and is visually observable from the exterior.

27. The container of claim 26, wherein said calcium ion chelator is capable of undergoing a color change upon exposure to calcium.

28. The container of claim 27, wherein said calcium ion chelator is arsenazo III or 3,3′-bis[N,N-di(carboxymethyl)aminomethyl]-o-cresolphthalein.

29. The container of claim 26, further comprising a magnesium ion chelator.

30. The container of claim 29, wherein said magnesium ion chelator is 8-hydroxyquinoline or 8-hydroxyquinoline-5-sulfonate.

31. The container of claim 26, wherein said potassium ion chelator is capable of undergoing a color change upon exposure to potassium.

32. The container of claim 31, wherein said second region further comprises a pH sensitive dye that undergoes a color change upon proton release.

33. The container of claim 32, wherein said potassium ion chelator is selected from the group consisting of valinomycin and 2,3-(naphtho)-15-crown-5.

34. The container of claim 33, wherein said pH sensitive dye is 4-[(2,6-dibromo-4-nitrophenyl)azo]-2-octadecyloxy-1-naphthol.

35. A kit comprising the tube of claim 17 and a color guide calibrated to indicate the concentration of calcium based on a comparison with the calcium-sensitive test strip.

36. The kit of claim 35, wherein said color guide comprises a color gradient corresponding to at least three distinct calcium concentration ranges.

37. The kit of claim 36, wherein said color guide is calibrated to indicate calcium concentrations of less than about 1 mg/dL, between about 5 mg/dL and about 7 mg/dL, and greater than about 7 mg/dL.

38. A method for evaluating an aliquot of a blood sample to determine the presence or identity of an additive that effects the level of at least one ion in the blood sample, comprising determining the concentration of one or more ions in said aliquot and identifying the additive based on the concentration of said one or more ions.

39. The method of claim 38, wherein said one or more ions comprises a calcium ion.

40. The method of claim 39, wherein the additive is identified as EDTA when said concentration of said calcium ion is less than 1 mg/dL.

41. The method of claim 40, wherein said concentration of said calcium ion is determined using a calcium ion chelator.

42. The method of claim 41, wherein said calcium ion chelator is arsenazo III or 3,3′-bis[N,N-di(carboxymethyl)aminomethyl]-o-cresolphthalein.

43. The method of claim 42, wherein said method further comprises contacting the aliquot with a magnesium ion chelator.

44. The method of claim 43, wherein said magnesium ion chelator is 8-hydroxyquinoline or 8-hydroxyquinoline-5-sulfonate.

45. The method of claim 41, wherein said method further comprises determining the concentration of potassium in said aliquot.

46. The method of claim 45, wherein the additive is identified as EDTA when said concentration of said calcium ion is less than 1 mg/dL and the concentration of said potassium is greater than about 20 mM.

47. The method of claim 38, wherein said one or more ions comprises a potassium ion.

48. The method of claim 47, wherein the aliquot is identified as being either additive-free or comprising heparin when said concentration of potassium is less than about 7 mM.

49. The method of claim 47, wherein the additive is identified as citrate when said concentration of potassium is about 7-20 mM.

50. The method of claim 47, wherein said concentration of potassium is determined using a potassium ion chelator.

51. The method of claim 50, wherein said potassium ion chelator is valinomycin or 2,3-(naphtho)-15-crown-5.
